(19) United States
(12) Patent Application Publication
(10) Pub. No.: US 2003/0185892 A1
(43) Pub. Date: Oct. 2, 2003

(54) Intraocular delivery compositions and methods

(76) Inventors: Steve J. D. Bell, Marietta, GA (US); Qing He, Atlanta, GA (US); Teh-Ching Chu, Atlanta, GA (US); David E. Potter, Sautee-Nacoochee, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/306,062

(22) Filed: Nov. 27, 2002

Related U.S. Application Data
(63) Continuation-in-part of application No. 09/932,538, filed on Aug. 17, 2001.

(51) Int. Cl. A61K 31/7048; A61K 39/395; A61K 38/47; A61K 31/65; A61K 31/4709; A61K 31/56

(52) U.S. Cl. 424/489; 424/130.1; 514/152; 424/94.61; 514/192; 514/29; 514/26; 514/308; 514/169; 514/534; 514/649

(57) ABSTRACT

The present invention relates to intraocular drug delivery for treating ocular diseases. Particularly, the invention relates to particles useful for the delivery of certain pharmacologically active agents to treat ocular diseases. The particles contain calcium phosphate core particles, particularly nanoparticles, as delivery agents and adjuvants. The invention also relates to methods of making such particles and to methods of treating ocular disease by delivery of a therapeutic drug to an ocular surface using the particles of this invention. The invention further relates to methods of regulating ocular pressure using certain formulations according to the present invention.

This application is a continuation-in-part of U.S. patent application Ser. No. 09/932,538 filed on Aug. 17, 2001, which claims benefit of the filing date of U.S. patent application Ser. No. 09/496,771 filed on Feb. 3, 2000, now issued as U.S. Pat. No. 6,355,271 B and which claims benefit of the filing dates of U.S. Provisional Application Serial Nos. 60/118,356; 60/118,364; and 60/118,355, all filed Feb. 3, 1999, the entire contents of each of which are hereby incorporated by reference.

### BACKGROUND OF INVENTION

1. Field of the Invention

The present invention relates to intraocular drug delivery for treating ocular diseases. Particularly, the invention relates to particles useful for the delivery of certain therapeutic agents to treat ocular diseases. The particles contain calcium phosphate core particles, particularly nanoparticles, which function as delivery agents and adjuvants. The invention also relates to methods of making such particles and to methods of treating ocular disease by delivery of a therapeutic drug to an ocular surface using the particles of this invention. The invention further relates to methods of regulating ocular pressure using certain formulations according to the present invention.

2. Description of Related Art

In the United States, 80 million people are currently afflicted with eye diseases that could result in blindness. In addition, 6.4 million cases of eye disease arise each year. Unfortunately, the number of Americans at risk for age-related eye diseases is increasing and is expected to double within the next three decades. Examples of the most common eye diseases include cataracts, corneal disease, diabetic retinopathy, macular degeneration, retinitis pigmentosa, retinoblastoma, strabismus, uveitus, and glaucoma.

A major problem in the treatment of eye diseases and disorders is the difficulty in delivering therapeutic agents into the eye at therapeutically effective concentrations. Oral administration of ocular drugs frequently results in undesired systemic side effects. In order for an effective amount of a therapeutic agent to reach the ocular area, a high concentration of drug must frequently be administered. This can result in systemic toxicity. Subcutaneously or intramuscularly administered alpha-interferon in adults may result in complications such as flu-like symptoms with fatigue, anorexia, nausea, vomiting, thrombocytopenia, and leukopenia.

There are also problems associated with the currently practiced methods of topical administration of ocular drugs. Topical administration is generally only effective in treating conditions involving the superficial surface of the eye and diseases which involve the cornea and anterior segments of the eye. Currently practiced methods of topical administration of drugs are ineffective in achieving adequate concentrations of drug in the sclera, vitreous, or posterior segment of the eye. In addition, topical administration is even less effective when the drug is a protein or peptide which typically lack the ability to cross the cornea.

Other available methods of ocular drug delivery include the use of topical or extraocular inserts. Neither, however, are highly desirable. Topical inserts are generally self-administered (resulting in the necessity of educating patients on insertion and removal), tend to fall out because of lid laxity, and are subject to the patient's lack of manual dexterity during self-treatment. In addition, topical inserts are generally only effective to deliver drug to the cornea and anterior chamber.

Extraocular inserts also have disadvantages. Frequent re-application is necessary because the therapeutic compound dissolves in a matter of hours. Again, these inserts only deliver drug to the cornea and anterior chamber.

Glaucoma is a disease for which an improved method of treatment is desired. Glaucoma is the leading cause of irreversible blindness in the world and is responsible for 5,500 new cases of blindness each year in the United States alone. A major risk factor in glaucoma is the elevation of intraocular pressure (IOP). The lens and cornea of an eye are nourished by a clear aqueous humor that circulates around the lens, through the pupil, and throughout the anterior chamber. Elevated IOP is the result of inadequate outflow of aqueous humor from the eye.

Treatment for glaucoma, and eye diseases in general, is often hindered by the efficacy of the available methods of treatment. As explained above, orally administered drugs are often associated with systemic side effects and topically administered drugs must be highly concentrated to counteract the brief contact time with the affected area and the resistance of the strong protective barrier of the eye.

Treatment for glaucoma usually begins with the administration of a topical drug. These drugs fall into five classes: β-adrenergic antagonists, prostaglandin analogues, adrenergic agonists, carbonic anhydrase inhibitors, and cholinergic agonists. Topical β-adrenergic antagonist drugs, such as Timolol maleate (marketed as Timoptic® by Merck), are most often used because of their efficiency in lowering IOP, their long duration of action, and few ocular side effects. However, this class of drug can lead to systemic side effects such as bronchospasm, respiratory failure, hypotension, and bradycardia.

Disadvantages of treatment with prostaglandin analogues include limited availability (currently only available as Pharmacia's Xalatan®) and systemic side effects including muscle and joint pain, allergic reactions of the skin, and the darkening of the color of the iris.

Andrenergic-agonist drugs (such as Allergan's Alphagan®) cause a decrease in the production of aqueous humor by constricting the vessels supplying the ciliary body and decreasing ultrafiltration. Side effects associated with these drugs include dry nose and dry mouth, systemic hypotension, and lethargy.

Carbonic anhydrase inhibitors decrease intraocular pressure by decreasing bicarbonate production and the flow of water into the posterior chamber of the eye. Although efficient, these drugs are often avoided because of their historical association with blood dyscrasia.

Finally, cholinergic agonists can also be used for the treatment of glaucoma. Cholinergic agonists work by stimulating parasympathetic receptors at neuromuscular junctions. Primary side effects include fixed, small pupils, induced myopia and a substantial risk of cataract inducement.

The dopamine D₂/D₃ receptor agonist, 7-hydroxy-dipropyl-aminotetralin (7-OH-DPAT), is another compound which recent research has found to be useful in the treatment of glaucoma. It has been shown to decrease both IOP and aqueous humor flow. Dopamine is an established major neurotransmitter in the central nervous system and retina. D₂/D₃ receptors located on the terminals of postganglionic sympathetic nerves in the anterior segment of the eye are thought to play a role in the suppression of aqueous humor formation and, thus, the level of intraocular pressure.

A significant problem with the above-mentioned drug therapies is the difficulty in delivering them to the entire ocular area with great efficacy. Topical drugs applied to the cornea must permeate the entire eye in order to reach the ocular area where the D₂/D₃ receptors are found. It has been proposed that ocular drugs delivered via conventional topical administration routes interact in some way with pigment, resulting in a decrease in the drug's desired activity.

Research has demonstrated that pigmented rabbits used in drug experimental models are generally less responsive to some ocular agents than their albino counterparts. In one study, medetomidine (MED), an alpha-2 agonist, and HA-118 (HA), a DA₂ agonist, were topically administered to both pigmented and albino rabbits. While MED was effective at lowering IOP in both strains of rabbits, HA was effective only in the albino variety. The researchers theorized that the lack of activity by HA in the pigmented variety arose from an absence of DA₂ receptors or excessive binding of HA to pigment in the anterior segment of the eyes. See Ogidigben et al., J. of Ocular Pharmacology, vol. 9, no. 3 (1993). Further research using raclopride has confirmed the presence of D₂/D₃ receptors on the postganglionic sympathetic nerves in the ciliary body and their participation in IOP regulation. See Chu et al., J. of Pharmacology and Experimental Therapeutics, vol. 293, no. 3 (2000).

Accordingly, there is a need for a delivery system for ocular drugs that reaches all segments of the eye and that discourages or avoids the binding of the pharmacologically active agent with pigment. The lack of efficacy in current ocular drug administration results in the use of more drug and more frequent administration in order to achieve optimal delivery concentrations. A delivery system which is more effective at dispersing the drug throughout the eye, using less drug and lasting for a longer duration, is thus highly desirable.

Recently, researchers have studied methods and compositions for delivering drugs across a mucosal surface such as the vagina, eye or nose. See U.S. Pat. No. 5,204,108. This reference describes microspheres between 10 and 100 microns that gel in contact with a mucosal surface. There are markedly different formulations for microspheres for drug delivery and as vaccine adjuvants, however, and there has been no indication that the drug delivery formulation has potential to elicit undesirable immune responses. Comparative studies have indicated that microparticles are potent adjuvants for mucosal delivery. However, microparticles are not in an ideal size range for inducing cellular immunity since they traditionally have been too large, and it is believed that dendritic cells and macrophages can more easily take up smaller particles. Nanometer scale particles have been proposed for use as carrier particles, as supports for biologically active molecules, such as proteins, and as decoy viruses. See U.S. Pat. Nos. 5,178,882; 5,219,577; 5,306,508; 5,334,394; 5,460,830; 5,460,831; 5,462,750; and 5,464,634, the entire contents of each of which are incorporated herein by reference.

The particles disclosed in the above-referenced patents, however, are generally extremely small, in the 10-200 nm size range. Particles of this size can be difficult to make with consistency. Moreover, these patents do not disclose the use of nanoparticles as controlled release matrices, and in particular, do not disclose the use of calcium phosphate particles as controlled release matrices for delivery of bioactive pharmaceuticals.

Earlier scientific reports have suggested a use for calcium phosphate (CAP) particles as adjuvants, but those calcium phosphate particles have generally been considered an unsuitable alternative to other adjuvants due to alleged inferior adjuvanting activity. See, e.g., Goto et al., Vaccine, vol. 15, no. 12/13 (1997). One of the more important distinctions between the previously-studied calcium phosphate particles and those of the present invention is that the chemical compositions and physical characteristics of the former calcium phosphate particles is markedly different from the particles of the present invention—hence, the former's inferior adjuvanting activity. Moreover, the calcium phosphate evaluated in Goto was typically microparticulate (>1000 nm diameter), amorphous, and possessed a rough and oblong morphology, in contrast to the more crystalline core particles of the present invention.

PCT Application No. WO 00/15194 to Lee et al. published on Mar. 23, 2000 also discusses calcium phosphate particles for delivery vehicles and adjuvants. This reference does not provide an adequate description of the use of its particle as a mucosal adjuvant, vaccine, or drug delivery agent. Moreover, the particles of the this reference would be difficult to manufacture because the method involves multiple steps and is thus far more time-consuming, labor-intensive, and costly.

Therefore, an important need remains for calcium phosphate core particles that can be effectively used as ocular adjuvants, as cores or carriers for biologically active molecules, as controlled release matrices, and as delivery vehicles for delivering pharmacologically active agents across ocular mucosal surfaces. For a number of therapeutic agents, delivery of the agent to a patient in need thereof can be difficult. Although topical administration is a viable option, currently available methods of topical ocular treatment often require long contact time with the cornea and frequent administration of the drug because of the cornea's resistance to penetration by foreign substances. The corneal epithelium and endothelium are lipid permeable, while the corneal stroma is water permeable. In order for corneal penetration to occur, the ocular drug must contain both water and lipid soluble particles. In addition, even after achieving corneal permeation, it is difficult to achieve adequate concentrations of drug in the sclera, vitreous, or posterior segments of the eye. The kinetics of ocular fluids work against the penetration of topically administered drugs from the cornea into the posterior segment of the eye. The above-mentioned research has demonstrated the tendency for many ocular drugs to bind to pigment in the anterior segment of the eye, preventing the drug molecules from reaching their targeted site. Furthermore, tears begin to rapidly wash the drug away before penetration into the cornea.

Despite the above-described attempts to provide effective treatment, there remains a long-felt and acute need for the design and development of efficacious therapeutic agents and methods of treatment for ocular diseases. The present inventors have developed a method of delivery of pharmacologically active agents to the ocular area using CAP particles. The CAP particles of the present invention may be useful for delivering any pharmacologically active agent for the treatment of ocular disease to ocular surfaces. In one specific embodiment, the inventors have developed a 7-OH-DPAT and CAP formulation which induces a decrease in both ocular pressure and aqueous humor flow. Importantly, this formulation requires less drug and has a longer sustainability than other formulations designed for the same purpose.

### SUMMARY OF THE INVENTION

The present invention relates to particles useful for the treatment of ocular diseases. The particles contain calcium phosphate and a pharmacologically active agent which is beneficial in the treatment of an ocular disease. The pharmacologically active agent is at least partially coating the particle or impregnating the particle or both. The present invention further relates to methods for treating ocular disease through delivery of the particles to the ocular surface of a patient in need thereof.

More specifically, the present invention relates to a unique formulation of calcium phosphate nanoparticles (CAP) for use in an ocular drug delivery system. The present inventors have found that CAP-based formulations of ocularly delivered drugs exhibit significantly increased efficacy rates. Although the exact mechanism of the CAP action is not fully understood, and while not wishing to be bound to any theory, it is thought that the CAP in vehicle with the drug formulation reduces the drug's ability to bind with pigment, thus enhancing the drug's desired activity.

Non-limiting examples of ocular diseases and disorders that may be treated by various embodiments of the present invention include glaucoma, uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

CAP is non-toxic, non-immunogenic, and is easily degraded by the body, and accordingly, CAP can be safely administered, and administration can be repeated using the same CAP vehicle for the same or different therapeutic agents. Moreover, the CAP particles of the present invention can be prepared relatively rapidly and inexpensively.

The present invention also relates to methods of preparing the novel calcium phosphate core particles having a pharmacologically active agent at least partially coated on the surface (“outside formulation”), impregnated therein (“inside formulation”), or both (“inside/outside formulation”).

The above discussed and many other features and attendant advantages of the present invention are detailed below. Other features and advantages of the invention will be apparent from the following description of the preferred embodiments thereof.

### BRIEF DESCRIPTION OF THE FIGURES

FIG. 1 is a schematic drawing showing a calcium phosphate core particle (4) both coated with a pharmacologically active agent (8) and having pharmacologically active agent (8) impregnated therein.

FIG. 2 is a series of schematic drawings showing various embodiments of calcium phosphate core particles. FIG. 2A shows a core particle coated directly with pharmacologically active agent (6). FIG. 2B shows a core particle (4) coated with surface modifying agent (2), such as polyethylene glycol or cellobiose, and having a pharmacologically active agent (6) adhered to the surface modifying agent (2). FIG. 2C shows a calcium phosphate core particle (4) having a surface modifying agent (2), such as polyethylene glycol or cellobiose incorporated therein and having a pharmacologically active agent (6) at least partially coating core particle (4).

FIG. 3 is a schematic drawing showing a calcium phosphate core particle (4) having both a surface modifying agent (2), such as polyethylene glycol or cellobiose and a material (6), such as a pharmaceutically active agent incorporated therein.

FIG. 4 shows the results from an experiment conducted to determine the effects on intraocular pressure in non-pigmented rabbits resulting from intraocular delivery of CAP alone, 7-OH-DPAT alone, and 7-OH-DPAT combined with CAP.

FIG. 5 shows the results from an experiment conducted to determine the effects on intraocular pressure in pigmented rabbits resulting from intraocular delivery of CAP alone, 7-OH-DPAT alone, and 7-OH-DPAT combined with CAP at three dosage levels.

FIG. 6 shows the results from an experiment conducted to determine the effects on intraocular pressure in pigmented rabbits resulting from intraocular delivery of the D₂/D₃ receptor antagonist raclopride alone, CAP alone, 7-OH-DPAT alone, 7-OH-DPAT combined with CAP, and 7-OH-DPAT combined with CAP and raclopride.

FIG. 7 shows the results of an experiment conducted to determine the effects on aqueous humor flow resulting from intraocular delivery of 7-OH-DPAT combined with CAP.

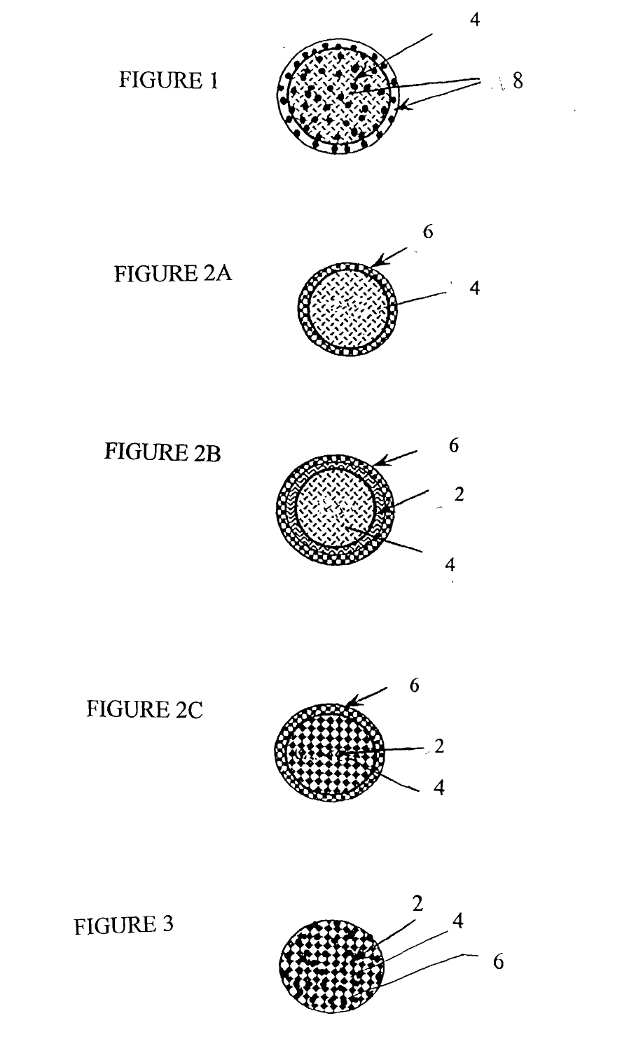

### DETAILED DESCRIPTION OF SPECIFIC EMBODIMENTS OF THE INVENTION

The present invention relates to novel calcium phosphate core particles for ocular delivery, to methods of making them, and to methods of using the core particles as cores or carriers for pharmacologically active native and recombinant agents, and as controlled release matrices for pharmacologically active native and recombinant agents. The present invention also relates to the novel calcium phosphate core particles for ocular delivery having a pharmacologically active agent at least partially coated on the surface of the core particles (“outside formulation”), or dispersed or impregnated within the core particles (“inside formulation”) or both (“outside/inside formulation”), to methods of making them, and to methods of using them.

One embodiment of the invention directed to a therapeutic agent in vehicle with CAP can be used for the treatment of any ocular disease, including, but not limited to, glaucoma, uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

Non-limiting examples of pharmacologically active agents within the scope of this invention to be at least partially coated on the surface of the core particle, impregnated therein, or both, include therapeutic proteins or peptides or other components capable of having a therapeutic effect when administered to an ocular surface.

Non-limiting examples of therapeutic agents which may be administered through the present invention include antibiotics, anti-angiogenic factors, anti-inflammatory factors, and neutrophic factors. Exemplary pharmacologically active agents for delivery using the particles of the present invention may also include antimicrobial agents, therapeutic monoclonal antibodies, such as tetracycline hydrochloride, leucomycin, penicillin, penicillin derivatives, erythromycin, sulphathiazole and nitrofurazone; local anesthetics such as benzocaine; vasoconstrictors such as phenylephrine hydrochloride, tetrahydrozoline hydrochloride, naphazoline nitrate, oxymetazoline hydrochloride and tramazoline hydrochloride; cardiotonics such as digitalis and digoxin; vasodilators such as nitro-glycerine and papaverine hydrochloride; antiseptics such as chlorhexidine hydrochloride, hexylresorcinol, dequaliniumchloride and ethacridine; enzymes such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

Examples of therapeutic agents which can be delivered by the present invention include gentamicin, idoxuridine, silver nitrate, tetracycline, prenisolone, tetracaine, acetazolamide, pilocarpine, timolol, atropine, epinephrine, brimonidine tartrate, levobunolol HCl, betaxolol GCl, dorzolamide- timolol, apraclonidine HCl, methazolamide, dipivefrin, unoprostone isopropyl, and latanoprost.

In addition to the CAP and a therapeutic agent, compositions of the present invention may include other components. For example, pharmaceutically acceptable buffers, preservatives, nonionic surfactants, solubilizing agents, stabilizing agents, emollients, lubricants and/or tonicity agents may be included. The compositions of the present invention may be delivered via a spray, an aerosol, an ointment, an eye drop, a gel, and so on. Those skilled in the art will understand how to formulate such vehicles by known techniques.

The core particles of the present invention may optionally have at least a partial coating of a surface modifying agent, which may help adhere the above-mentioned therapeutic agent to the core particle, or may have a surface modifying agent impregnating the particle, or both. A further aspect of the invention provides a method of treatment by administering a formulation as described above to an ocular surface.

I. Core Particles

The calcium phosphate core particles of the present invention have an average particle size between about 300 nm and about 4000 nm, more particularly, between about 300 nm and about 2000 nm. For the applications described herein, an average particle size of between about 300 nm and 1000 nm is sufficient and desirable. The core particles of the present invention have a morphology that is generally and substantially spherical in shape and a surface that is substantially smooth.

The term “substantially smooth” is used herein to mean essentially no surface features or irregularities having a size of 100 nm or larger. The core particles may be faceted or angular and still fall within this definition, as long as the facets do not contain many surface irregularities of the type described above. The term “substantially spherical” is used herein to refer to particles that are substantially round or oval in shape, and includes particles that are unfaceted and smooth, or that have very few facets, as well as particles that are polyhedral having several or numerous facets.

The following table provides a comparison between the calcium phosphate core particles of the present invention and calcium phosphate particles manufactured by Superfos Biosector a/s. The table shows that the calcium phosphate core particles of the present invention are small, smooth and ovoid, whereas Superfos Accurate CAP particles are large, amorphous, jagged and crystalline.

| | BioSante | |
|---|---|---|
| | Pharmaceuticals, Inc. | Superfos Biosector a/s |
| | CAP | CAP |
| PH | 6.2-6.8 | 6.49 |
| Size | <1000 nm | >3000 nm |
| Morphology | Smooth ovoid shape | Jagged crystalline shape |

The calcium phosphate core particles of the present invention are typically prepared as a suspension in aqueous medium by reacting a soluble calcium salt with a soluble phosphate salt, and more particularly, by reacting calcium chloride with sodium phosphate under aseptic conditions. Initially, an aqueous solution of calcium chloride having a concentration between about 5 mM and about 300mM is combined by mixing with an aqueous solution of a suitable distilled water-based solution of sodium citrate, having a concentration between about 5 mM and about 300 mM. The presence of sodium citrate contributes to the formation of an electrostatic layer around the core particle, which helps to stabilize the attractive and repulsive forces between the core particles, resulting in physically stable calcium phosphate core particles.

An aqueous solution of dibasic sodium phosphate having a concentration between about 5 mM and about 300 mM is then mixed with the calcium chloride/sodium citrate solution. Turbidity generally forms immediately, indicating the formation of calcium phosphate core particles. Mixing is generally continued for at least about 48 hours, or until a suitable core particle size has been obtained, as determined by sampling the suspension and measuring the core particle size using known methods. The core particles may be optionally stored and allowed to equilibrate for about seven days at room temperature to achieve stability in size and pH prior to further use.

In one embodiment, the core particles of the present invention can also be at least partially coated or impregnated or both with a pharmacologically active agent, wherein the pharmacologically active agent is disposed on the surface of the core particle and optionally held in place by a surface modifying agent sufficient to bind the material to the core particle without denaturing the material. Non-limiting examples of the pharmacologically active agent are discussed above.

In a further embodiment, the particles are complexed with surface modifying agents suitable for use in the present invention include substances that provide a threshold surface energy to the core particle sufficient to bind material to the surface of the core particle, without denaturing the material. Example of suitable surface modifying agents include those described in U.S. Pat. Nos. 5,460,830, 5,462,751, 5,460,831, and 5,219,577, the entire contents of each of which are incorporated herein by reference. Non-limiting examples of suitable surface modifying agents may include basic or modified sugars, such as cellobiose, or oligonucleotides, which are all described in U.S. Pat. No. 5,219,577. Suitable surface modifying agents also include carbohydrates, carbohydrate derivatives, and other macromolecules with carbohydrate-like components characterized by the abundance of -OH side groups, as described, for example, in U.S. Pat. No. 5,460,830. Polyethylene glycol (PEG) is a particularly suitable surface modifying agent.

The core particles may be at least partially coated by preparing a stock solution of a surface modifying agent, such as cellobiose or PEG (e.g., around 292 mM) and adding the stock solution to a suspension of calcium phosphate core particles at a ratio of about 1 mL of stock solution to about 20 mL of particle suspension. The mixture can be swirled and allowed to stand overnight to form at least partially coated core particles. The at least partially coated core particles are administrable alone or in conjunction with one or more of the materials described below. Generally, this procedure will result in substantially complete coating of the particles, although some partially coated or uncoated particles may be present.

II. Pharmacologically Active Agent

The core particles described above are adapted to support a pharmacologically active agent. The calcium phosphate core particles of the present invention can be prepared as controlled release particles for the sustained release of the pharmacologically active agent over time, wherein the pharmacologically active agent is incorporated into the structure of the core particle or coated on the outside, or both.

A. Coating

Coating of the core particles with a pharmacologically active agent is preferably carried out by suspending the core particles in a solution containing a dispersed surface modifying agent, generally a solution of double distilled water containing from about 0.1 to about 30 wt % of the surface modifying agent. The cores are maintained in the surface modifying agent solution for a suitable period of time, generally about one hour, and may be agitated, e.g., by rocking or sonication. The at least partially coated core particles can be separated from the suspension, including from any unbound surface modifying agent, by centrifugation. The at least partially coated core particles can then be resuspended in a solution containing the pharmacologically active agent to be adhered to the at least partially coated core particle. Optionally, a second layer of surface modifying agent may also be applied to the pharmacologically active agent adhered to the particle.

In another embodiment, a pharmacologically active agent may be attached to an unmodified particle surface, although particles at least partially coated with a surface modifying agent have greater loading capacities. For example, loading capacities of at least partially coated particles have been found to be about 3 to 4-fold higher than loading capacities of unmodified particle surfaces. Additionally, an increase in particle size may result in a greater loading capacity. For instance, an increase of 150 nm in particle size (relative to a starting size of 450 nm to 600 nm) results in about a 3-fold increase in loading capacity in particles that are at least partially coated with a surface modifying agent.

Another embodiment that facilitates higher loading capacities is schematically illustrated in FIG. 2C, which shows a core particle having a surface modifying agent (2), such as polyethylene glycol, impregnated therein. The particles may be prepared by adding a surface modifying agent (2) to one or more of the aqueous solutions forming the core particle (4). The core particles may optionally be stored at room temperature. To obtain at least partially coated particles, the particles are subsequently contacted with a pharmacologically active agent, such as an antibiotic, to provide at least a partial coating on the particle as described above.

B. Impregnated

A further embodiment facilitating higher loading capacities is illustrated in FIG. 3, which shows a core particle (4) having both a surface modifying agent (2), such as polyethylene glycol, and a material (6), such as a pharmacologically active agent, impregnated therein. One way in which particles of this embodiment may be prepared is by combining a desired pharmacologically active agent and a surface modifying agent together to form a solution. This solution is then combined with one or more of the aqueous solutions forming the particle as described above. The resulting particles incorporate calcium phosphate, surface modifying agent, and a pharmacologically active agent within the core particle. Particles prepared according to this and any other embodiments described herein may be combined with one or more particles prepared according to any other embodiment described herein.

Incorporating a pharmacologically active agent into the particle may be accomplished by mixing an aqueous calcium chloride solution with the therapeutic agent to be incorporated prior to combining and mixing with either the sodium citrate or dibasic sodium phosphate solutions, to co-crystallize the calcium phosphate core particles with the pharmacologically active agent.

The particles and pharmaceutical compositions of this invention may be suitably administered to any patient in need thereof, namely to any species of animal that suffers or can suffer from any eye disease or eye condition requiring treatment with any ocular drug, protein, or peptide, more particularly mammals, and even more particularly humans. Non-limiting examples of diseases or disorders which may be treated in this way include uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

Non-limiting examples of therapeutic agents which may be administered through the present invention include antibiotics, antimicrobial agents, therapeutic monoclonal antibodies, such as tetracycline hydrochloride, leucomycin, penicillin, penicillin derivatives, erythromycin, sulphathiazole and nitrofurazone; local anesthetics such as benzocaine; vasoconstrictors such as phenylephrine hydrochloride, tetrahydrozoline hydrochloride, naphazoline nitrate, oxymetazoline hydrochloride and tramazoline hydrochloride; cardiotonics such as digitalis and digoxin; vasodilators such as nitro-glycerine and papaverine hydrochloride; antiseptics such as chlorhexidine hydrochloride, hexylresorcinol, dequaliniumchloride and ethacridine; enzymes such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

Generally, the inventors have found that the topical application of 7-OH-DPAT in pigmented rabbit eyes reduces intraocular pressure when combined with CAP but not when administered alone. These findings demonstrate that CAP delivered as a delivery system enhances activity by 7-OH-DPAT in pigmented rabbit eyes suggesting that CAP is useful for achieving controlled and targeted drug delivery for treatment of ocular disease.

The various embodiments of the invention can be more clearly understood by reference to the following nonlimiting examples.

### EXAMPLE 1

12.5 mM calcium chloride, 12.5 mM dibasic sodium phosphate and 15.6 mM sodium citrate were mixed together in water and stirred for 48 hours. After the reaction was completed, the suspension of CAP particles was sonicated with 550 Sonic Dismembrator (Fisher Scientific, Pittsburgh, Pa.) for 30 minutes and stored at room temperature. Nanoparticles were characterized by particle size utilizing a Laser Defractometer (Coulter® N4Plus). The size of particles ranged from 70 to 1075 nm with the majority of the particles in 701 nm and in 196 nm. The pH of the solution containing particles was 6.8 determined by a pH meter (Model AP15, Fisher Scientific, Pittsburgh, Pa.). Scanning electron microscopy also demonstrated the surface morphology. CAP was concentrated to 3 mg/ml by centrifugation at 7,000g for 15 minutes. To formulate the drug solutions, CAP were loaded with different doses of 7-OH-DPAT. 7-OH-DPAT was dissolved in 0.5 ml cellobiose solution (the concentrate of stock solution, 100 mg/ml) and aliquots of this solution were added to 0.5 ml CAP to provide the desired dosages. The mixture was rotated for 2 hours at room temperature before use.

### EXAMPLE 2

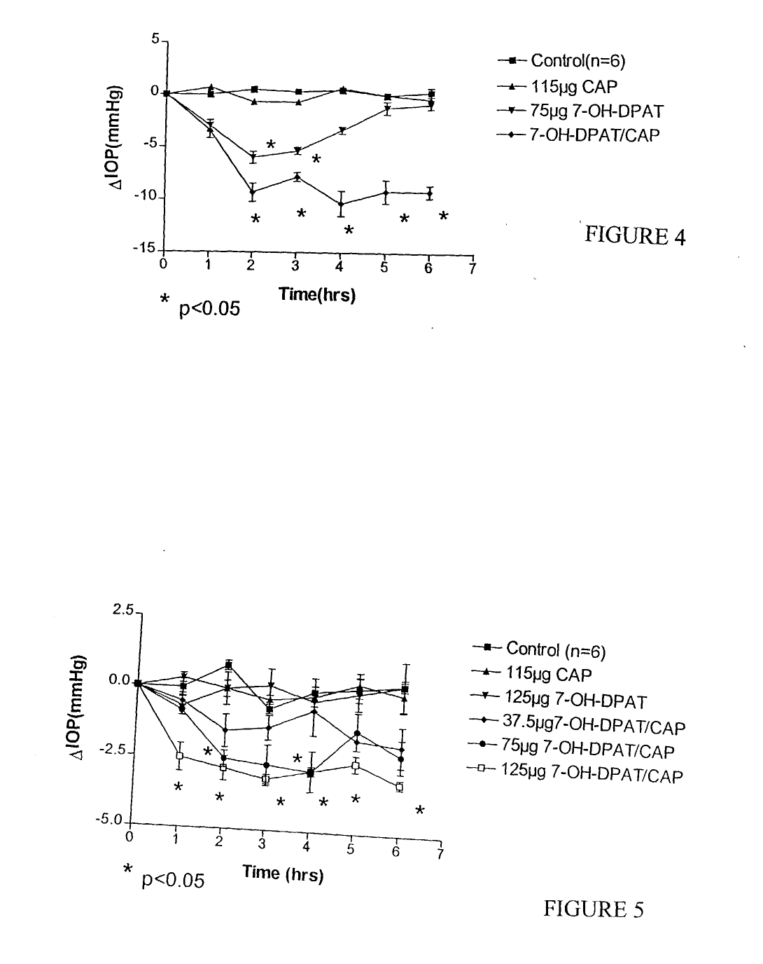

Previous studies reported that 7-OH-DPAT, a dopamine D₂/D₃ receptor agonist, produced dose-related IOP lowering effects in New Zealand White (NZW) rabbits. A comparative study was conducted in which the medium dose (75 μg) of 7-OH-DPAT was combined with CAP and administered to NZW rabbits. The results of the experiment are shown in FIG. 4. The results indicate that rabbits treated with CAP combined with 7-OH- DPAT exhibit lower intraocular pressure over time as compared to rabbits that either remained untreated or that were treated with CAP alone or with 7-OH-DPAT alone.

The particles that were used in this specific experiment were prepared as discussed in Example 1, but it should be understood that any of the methods described herein could be used to prepare effective particles. To test the efficacy of the particles, an effective amount of the 7-OH-DPAT in vehicle with CAP was delivered intraocularly to the eyes of rabbits and the eye pressure was checked. The ocular hypotension induced by topical administration of 7-OH-DPAT with CAP was more pronounced and sustained that that of 7-OH-DPAT alone or CAP alone.

### EXAMPLE 3

A study was conducted to investigate the efficacies of dose-related 7-OH-DPAT with CAP compared with 7-OH-DPAT alone on Dutch Belted (DB) pigmented rabbits.

The results of the experiment are shown in FIG. 5. Generally, pigmented rabbits treated with greater doses of 7-OH-DPAT combined with CAP exhibited lower intraocular pressure over time as compared to rabbits that either remained untreated or that were treated with CAP alone, with 7-OH-DPAT alone, or with lower dosages of 7-OH-DPAT with CAP.

The particles that were used in this specific experiment were prepared as discussed in Example 1, but it should be understood that any of the methods described herein could be used to prepare effective particles.

The addition of CAP caused a dose-proportional reduction in IOP that was pronounced and sustained, while 7-OH- DPAT alone had no effect

### EXAMPLE 4

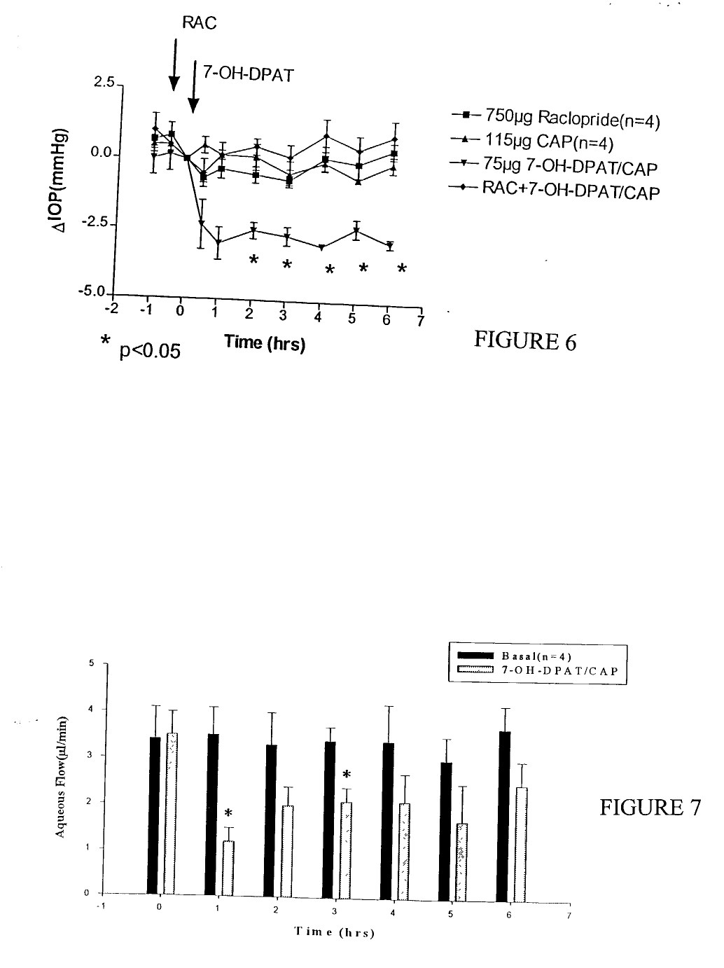

To confirm the involvement of a dopamine D₂/D₃ receptor mechanism in pigmented rabbits, experiments were performed in which pretreatment with a dopamine D₃/D₂ receptor antagonist, raclopride, was used to investigate the ocular hypotension by 7-OH-DPAT in vehicle with CAP. The results of the experiment are shown in FIG. 6. The results indicate that raclopride treatment alone did not change intraocular pressure; however, pretreatment with 750 μg of raclopride inhibited the ocular hypotension induced by 75 μg of 7-OH-DPAT in vehicle with CAP. These results verify the role of dopamine D₃/D₂ receptors in 7-OH-DPAT in vehicle with CAP's ability to reduce IOP.

### EXAMPLE 5

An experiment was conducted to identify the potential mechanisms in which 7-OH-DPAT in vehicle with CAP induces ocular hypotension. The results are shown in FIG. 7. Aqueous humor flow rates were measured in both untreated DB rabbits and DB rabbits treated with 75 μg 7-OH-DPAT in vehicle with CAP. The results show that treatment with 7-OH-DPAT in vehicle with CAP resulted in bilateral decreases in both IOP and aqueous humor flow rates. These results indicate that 7-OH-DPAT in vehicle with CAP suppresses aqueous humor flow, thereby lowering IOP.

The particular embodiments of the invention having been described above are not limiting of the present invention, and those of skill in the art can readily determine that additional embodiments and features of the invention are within the scope of the appended claims and equivalents thereto.

What is claimed is:

1. A particle comprising calcium phosphate and a pharmacologically active agent at least partially coating the particle or impregnating the particle or both, wherein the particle is adapted to deliver the pharmacologically active agent to an ocular surface of a patient in need thereof for treatment of an ocular disease.

2. The particle of claim 1, further comprising a surface modifying agent, such as polyethylene glycol.

3. The particle of claim 1, further comprising a surface modifying agent, such as polyethylene glycol, wherein the pharmacologically active agent is located on the surface of the particle, impregnated in the particle, or both.

4. The particle of claim 1, wherein the pharmacologically active agent is a therapeutic drug used to treat glaucoma, uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

5. The particle of claim 1, wherein the pharmacologically active agent is an antibiotic, an antimicrobial agent, a therapeutic monoclonal antibody, such as tetracycline hydrochloride, leucomycin, penicillin, penicillin derivatives, erythromycin, sulphathiazole and nitrofurazone; a local anesthetic such as benzocaine; a vasoconstrictor such as phenylephrine hydrochloride, tetrahydrozoline hydrochloride, naphazoline nitrate, oxymetazoline hydrochloride and tramazoline hydrochloride; a cardiotonic such as digitalis and digoxin; a vasodilator such as nitro-glycerine and papaverine hydrochloride; an antiseptic such as chlorhexidine hydrochloride, hexylresorcinol, dequaliniumchloride and ethacridine; an enzyme such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

6. The particle of claim 1, wherein the pharmacologically active agent is 7-hydroxy-2-dipropyl-aminotetralin.

7. The particle of claim 2, wherein the pharmacologically active agent is a therapeutic drug used to treat glaucoma, uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

8. The particle of claim 2, wherein the pharmacologically active agent is an antibiotic, an antimicrobial agent, a therapeutic monoclonal antibody, such as tetracycline hydrochloride, leucomycin, penicillin, penicillin derivatives, erythromycin, sulphathiazole and nitrofurazone; a local anesthetic such as benzocaine; a vasoconstrictor such as phenylephrine hydrochloride, tetrahydrozoline hydrochloride, naphazoline nitrate, oxymetazoline hydrochloride and tramazoline hydrochloride; a cardiotonic such as digitalis and digoxin; a vasodilator such as nitro-glycerine and papaverine hydrochloride; an antiseptic such as chlorhexidine hydrochloride, hexylresorcinol, dequaliniumchloride and ethacridine; an enzyme such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

9. The particle of claim 2, wherein the pharmacologically active agent is 7-hydroxy-2-dipropyl-aminotetralin.

10. A method for treating ocular disease, comprising delivering a particle of claim 1 to an ocular surface of the patient in need thereof, wherein the pharmacologically active agent is a therapeutic drug for treatment of ocular disease.

11. The method of claim 10, wherein the particle further comprises a surface modifying agent, such as polyethelene glycol.

12. The method of claim 10, wherein the pharmacologically active agent is a therapeutic drug used to treat glaucoma, uveitis, retinitis pigmentosa, macular degeneration, retinopathy, retinal vascular diseases, and other vascular anomalies, endophthalmitis, infectious diseases, inflammatory but non-infectious diseases, ocular ischemia syndrome, peripheral retinal degenerations, retinal degenerations, choroidal disorders and tumors, vitreous disorders, and inflammatory optic neuropathies.

13. The method of claim 10, wherein the pharmacologically active agent is an antibiotic, an antimicrobial agent, a therapeutic monoclonal antibody, such as tetracycline hydrochloride, leucomycin, penicillin, penicillin derivatives, erythromycin, sulphathiazole and nitrofurazone; a local anesthetic such as benzocaine; a vasoconstrictor such as phenylephrine hydrochloride, tetrahydrozoline hydrochloride, naphazoline nitrate, oxymetazoline hydrochloride and tramazoline hydrochloride; a cardiotonic such as digitalis and digoxin; a vasodilator such as nitro-glycerine and papaverine hydrochloride; an antiseptic such as chlorhexidine hydrochloride, hexylresorcinol, dequaliniumchloride and ethacridine; an enzyme such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

14. The method of claim 10, wherein the pharmacologically active agent is 7-hydroxy-2-dipropyl-aminotetralin.

15. A method for preparing particles suitable for the treatment of ocular disease, comprising:
(a) mixing an aqueous solution of calcium chloride with an aqueous solution of sodium citrate to form a mixture;
(b) adding an aqueous solution a sodium phosphate to the mixture to form a solution;
(c) stirring the solution until particles of the desired size and comprising calcium phosphate are obtained; and
(d) dissolving a therapeutic drug used for the treatment of ocular disease in cellobiose solution and adding this solution to the calcium phosphate particles to form particles that are at least partially coated and at least partially impregnated with the therapeutic drug.

16. The method of claim 15, wherein the stirring comprising sonicating.

17. The method of claim 15, wherein the therapeutic drug is 7-hydroxy-2-dipropyl-aminotetralin.

18. A particle comprising
(a) calcium phosphate, and
(b) a pharmacologically active agent selected from the group: antibiotics, antimicrobial agents, therapeutic monoclonal antibodies, such as tetracycline hydrochloride, leucomycin, penicillin, penicillin derivatives, erythromycin, sulphathiazole and nitrofurazone; local anesthetics such as benzocaine; a vasoconstrictor such as phenylephrine hydrochloride, tetrahydrozoline hydrochloride, naphazoline nitrate, oxymetazoline hydrochloride and tramazoline hydrochloride; cardiotonics such as digitalis and digoxin; a vasodilators such as nitro-glycerine and papaverine hydrochloride; antiseptics such as chlorhexidine hydrochloride, hexylresorcinol, dequaliniumchloride and ethacridine; enzymes such as lysozyme chloride and dextranase; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds at least partially coating the particle or impregnating the particle or both;
wherein the particle is adapted to deliver the pharmacologically active agent to an ocular surface of a patient in need thereof.
